(19) United States
(12) Patent Application Publication
(10) Pub. No.: US 2019/0082744 A1
(43) Pub. Date: Mar. 21, 2019

(54) UNDERGARMENT AND METHODS OF MAKING AND USING SAME

(71) Applicant: Heather Harley-LeGrand, Okmulgee, OK (US)

(72) Inventor: Heather Harley-LeGrand, Okmulgee, OK (US)

(21) Appl. No.: 16/135,696

(22) Filed: Sep. 19, 2018

Related U.S. Application Data
(60) Provisional application No. 62/560,461, filed on Sep. 19, 2017.

(51) Int. Cl. A41B 9/00; A41B 9/04; A61L 9/014

(52) U.S. Cl. CPC A41B 9/004; A41B 9/002; A41B 17/005; A61L 9/014; A41B 9/04

(57) ABSTRACT

An undergarment having a body portion, a channel and a tubular member. The body portion has a front portion, a back portion and a crotch portion positioned between the front portion and back portion wherein the crotch portion has a first end and a second end. The channel extends between a first end and a second end of the crotch portion. The tubular member is positioned in the crotch portion of the body portion of the undergarment so as to fit between the buttocks of an individual.

### CROSS REFERENCE TO RELATED APPLICATIONS/ INCORPORATION BY REFERENCE STATEMENT

The present patent application claims priority to U.S. Provisional Patent Application U.S. Ser. No. 62/560,461, filed on Sep. 19, 2017, the entire contents of which is hereby expressly incorporated herein by reference.

### FIELD OF THE INVENTION

The present disclosure relates generally to clothing and more particularly to undergarments for attenuating and/or lessening the sound that occurs from flatulence.

### BACKGROUND

Flatulence is a normal physiological occurrence in humans. However, making noise from flatulence in public can cause great embarrassment to an individual.

To this end, an undergarment is needed to eliminate or decrease the sound that occurs from flatulence. It is to such an undergarment and methods of making and using same that the present disclosure is directed.

### BRIEF DESCRIPTION OF THE DRAWING(S)

FIG. 1 is a perspective view of one embodiment of an undergarment constructed in accordance with the present disclosure, the undergarment positioned on an individual.

FIG. 2 is a front view of the undergarment of FIG. 1.

FIG. 3 is a back view of the undergarment of FIG. 1.

FIG. 4 is a crotch portion of the undergarment of FIG. 1.

FIG. 5 is a top view of another embodiment of an undergarment constructed in accordance with the present disclosure.

FIG. 6 is a cutaway view of a portion of the undergarment of FIG. 5.

### DETAILED DESCRIPTION

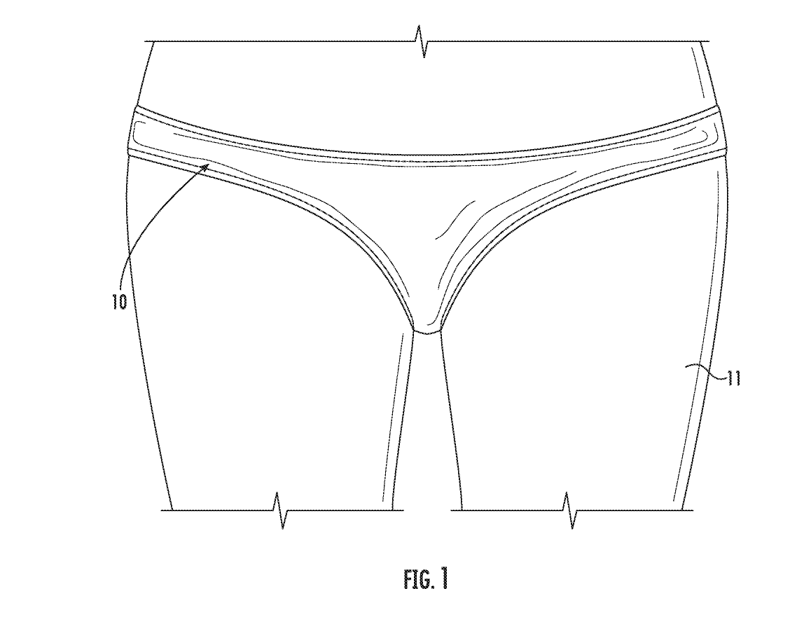

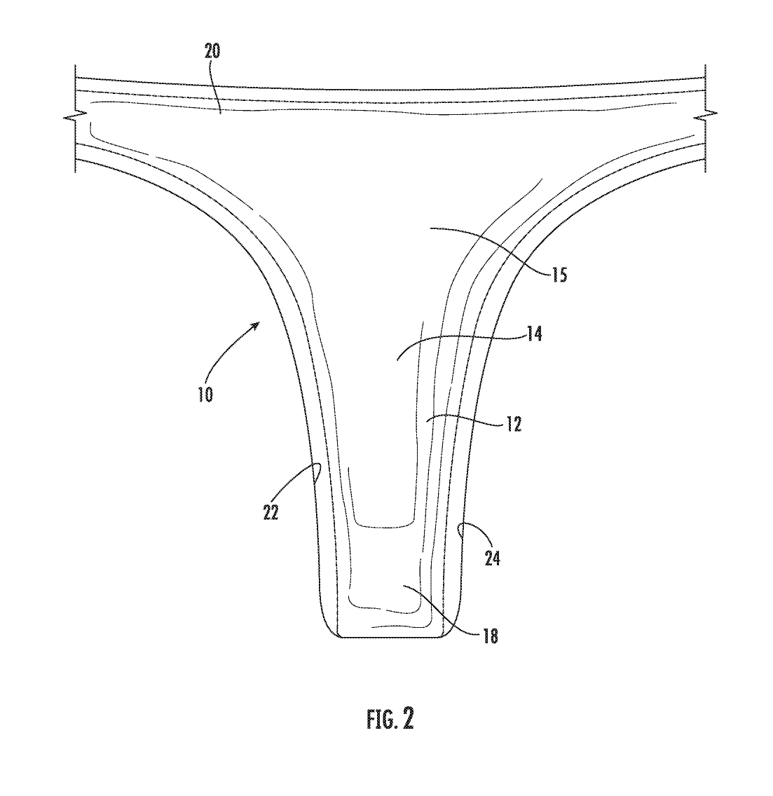

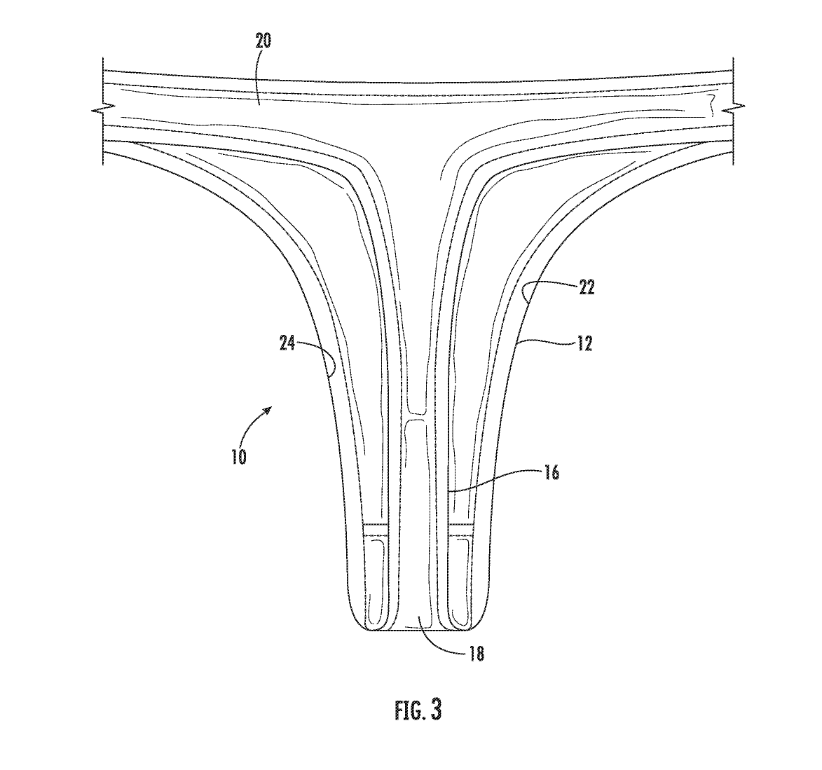

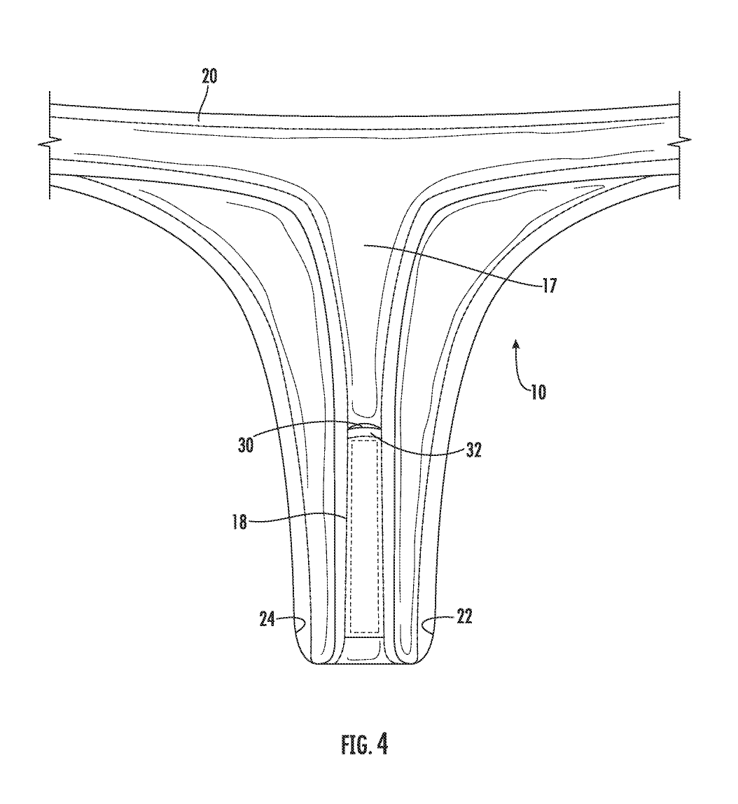

Referring now to the drawings, and in particular to FIGS. 1-4, shown therein and designated by a reference numeral 10, is an undergarment constructed in accordance with the present invention. In the embodiment illustrated herein, the undergarment 10 is a pair of female thong panties positioned on a female 11, however, the undergarment could take the form of any such garment used by male or female, such as underwear, underpants, briefs, shorts, swimsuits, long pants or any other item of apparel that covers the crotch of an individual. Preferably, the undergarment 10 is a pair of cotton or silk panties, or some other highly washable and durable fabric. It should be understood by one of ordinary skill in the art that the undergarment may be formed of virtually any material, elastic or otherwise. It is contemplated that the undergarment 10 is constructed from multiple pieces of material, however, it should be understood that the undergarment 10 may be constructed from one solid piece of material.

Broadly, in one embodiment, the undergarment 10 includes a body portion 12 having a front portion 14, a back portion 16 and a crotch portion 18. The body portion 12 has an outer surface 15 and an inner surface 17. The body portion 12 has a waistband 20 and leg openings 22 and 24.

The undergarment 10 further includes a sound attenuating member 30. The sound attenuating member 30 is supported by the crotch portion 18 adjacent the back of the crotch of the individual wearing the undergarment 10. As used herein, “crotch” refers generally to the area of the human body between the legs where the legs join the torso. In one embodiment, the sound attenuating member 30 is flexible so that it will conform to the shape between an individual's buttocks and bend when the wearer moves. More preferably, the sound attenuating member 30 is made from a sound absorbing material in order to absorb the sound of the wearer's flatulence during movement and activities. It should be understood that the sound attenuating member 30 is used to absorb flatulence whether the gas is expelled from the anus or air is expelled from the vagina.

As indicated, the sound attenuating member 30 is supported in the crotch portion 18 of the undergarment 10 and under a portion of the crotch of the individual. To this end, the crotch portion 18 of the undergarment 10 may be formed with a pocket or pouch 32 for inserting the sound attenuating member 30. Generally, the pocket 32 will be formed on the inside of the crotch portion 18 of the undergarment 10; however, a similar sound attenuating effect would be achieved by supporting the sound attenuating member 30 on the outside of the undergarment 10.

The pocket 32 may be simply provided by stitching a piece of fabric slightly larger than the sound attenuating member 30 on the inside of the crotch portion 18 of the undergarment 10. This fabric should be washable and non-abrasive, as it will directly contact the skin of the individual wearing the undergarment. It will be apparent now that in some cases, it may be advantageous to leave a portion of the pocket open so that the sound attenuating member 30 can be removed. To that end, the pocket could be provided with a zippered, hook and latch, buttoned or flapped closure. It should be understood that in some embodiments, the sound attenuating member 30 will not be removable.

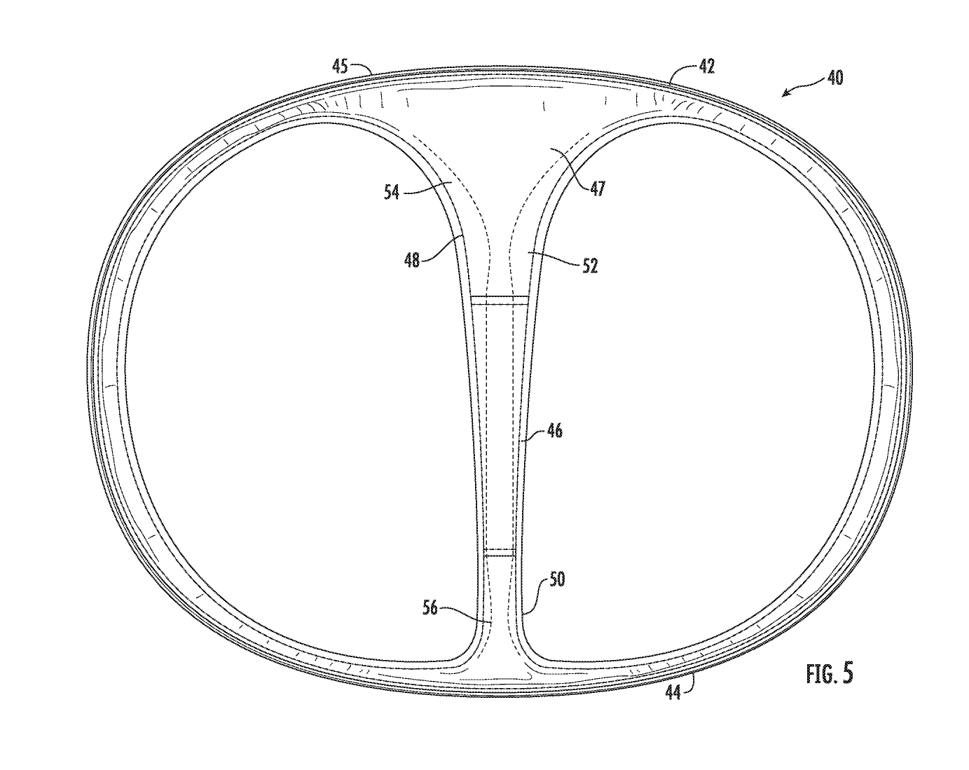

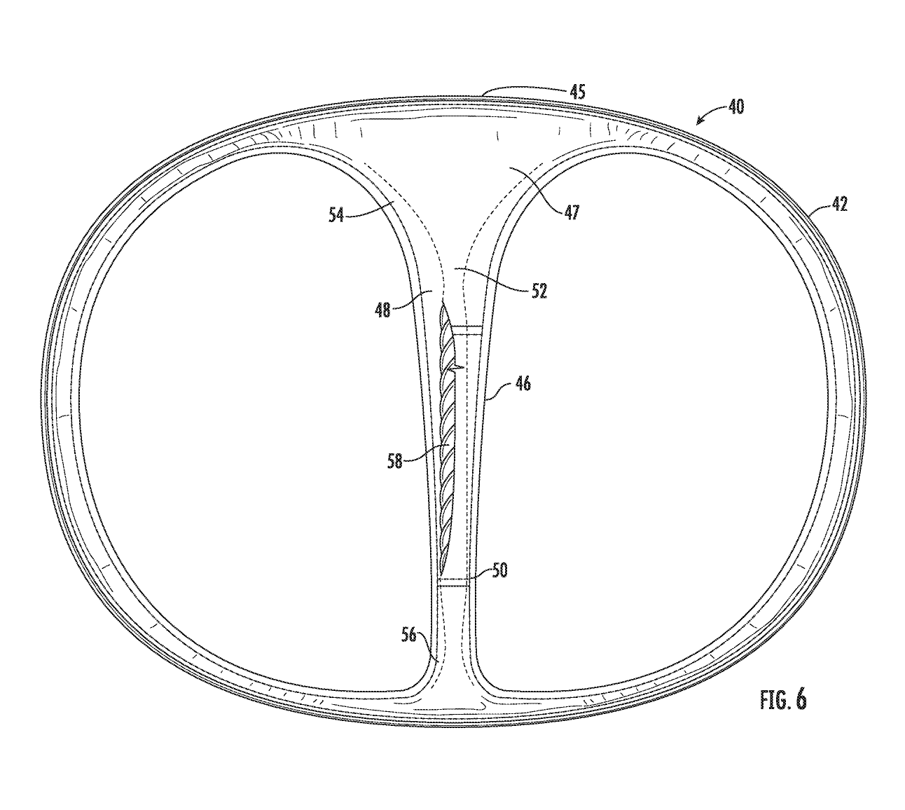

Referring now to FIGS. 5 and 6, another embodiment of an undergarment 40 is shown similar to the undergarment 10 except as described herein. The undergarment 40 includes a front portion 42, a back portion 44, a crotch portion 46, an outer surface 45 and an inner surface 47. The crotch portion 46 has a first end 48 and a second end 50 and a channel or passageway 52 extending between the first end 48 and the second end 50 of the crotch portion 46. In one embodiment, the channel 52 extends proximate the first end 48 and proximate the second end 50 of the crotch portion 46. In other embodiments, the channel 52 extends proximate the front portion 42 at one end and proximate the back portion 44 of the opposite end.

The channel 52 has a first end 54 and a second end 56. The channel 52 is sized and dimensioned to receive a tubular member 58. The channel 52 is formed about, on or in a portion of the inner surface 47 of the crotch portion 46. The first end 54 and/or second end 56 are each partially, substantially v-shaped and each substantially narrows at portions so as to hold the tubular member 58 in the channel 52 and keep the tubular member 58 from falling out of the channel 52. The tubular member 58 is movably held in a portion of the channel 52. The first end 54 and/or second end 56 is sized so that an individual can place their finger in the channel 52 so as to move the tubular member 58 to the desired position. It is contemplated that in some embodiments, the first end 54 and the second end 56 of the channel 52 may be opened and/or closed and combinations thereof.

The tubular member 58 is constructed from a material which is flexible, semi-rigid, rigid, or any combination thereof. The material comprises woven or unwoven fabric, synthetic or natural fabric, cardboard, fiber, elastomeric, cloth, burlap, or the like or combinations thereof. The tubular member 58 may be any size, shape and/or dimension depending on the needs of the individual using the undergarment 40. The tubular member 58 is positioned in the channel so as to be positioned between the buttocks so as to hold open the anus and/or between the labia of the vagina to release the air so as attenuate flatulence.

In use, an individual puts on the undergarments 10 and 40 like any method for putting on a conventional undergarment. The sound attenuating member 30 and tubular member 58 are each, respectively, positioned by the individual toward the back of the individual's crotch so as to fit between the buttocks of the individual and/or the labia of the vagina of an individual.

One of the main purposes of the undergarments 10 and 40 is to reduce and/or prevent flatulence sounds/vibrations and thus to prevent humiliation and embarrassment of the individual during daily activities, such as exercise (yoga, jogging, etc.), sleeping (at home or public places, airplanes, etc.) or for medical reasons (irritable bowel syndrome, or other digestive health conditions).

It should be understood that changes may be made in the operation and the construction of such embodiment.

It is important to note that the construction and arrangement of the undergarment as shown in the exemplary embodiment is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements and elements shown as multiple parts or may be constructed to be integrally formed, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present embodiments.

From the present description, it is clear that the present invention is well adapted to carry out the objects and to attain the advantages mentioned herein as well as those inherent in the invention. While presently preferred embodiments of the invention have been described for purposes of this disclosure, it will be understood that numerous changes may be made which will readily suggest themselves to those skilled in the art and which are accomplished within the spirit of the invention disclosed.

What is claimed is:

1. An undergarment, comprising:
a body portion having a front portion, a back portion and a crotch portion positioned between the front portion and back portion wherein the crotch portion has a first end and a second end;
a channel extending between a first end and a second end of the crotch portion; and
a tubular member positioned in the channel of the crotch portion of the body portion of the undergarment.

2. The undergarment of claim 1 wherein the crotch portion has an outer surface and an inner surface such that the channel is formed on the inner surface of the crotch portion.

3. The undergarment of claim 1 wherein the channel has a first end and a second end.

4. The undergarment of claim 3 wherein the first end of the channel is partially, substantially v-shaped.

5. The undergarment of claim 3 wherein the second end of the channel is partially, substantially v-shaped.

6. The undergarment of claim 3 wherein the first end and the second end of the channel narrow toward each other such that the tubular member is movably held in a portion of the channel.

7. The undergarment of claim 1 wherein the first end and the second end of the channel are each pratially substantialy v-shaped such that the tubular member is movably hel in a portion of the channel.

8. The undergarment of claim 1 wherein the tubular member is positioned between the buttocks of an individual wearing the undergarment.

9. The undergarment of claim 1 wherein the tubular member is positioned between the labia of an individual wearing the undergarment.

10. The undergarment of claim 1 wherein the tubular member is constructed of a flexible material.
